(19) United States
(12) Patent Application Publication
(10) Pub. No.: US 2024/0255444 A1
(43) Pub. Date: Aug. 1, 2024

(54) X-RAY TRANSMISSION INSPECTION APPARATUS AND X-RAY TRANSMISSION INSPECTION METHOD

(71) Applicant: HITACHI HIGH-TECH SCIENCE CORPORATION, Tokyo (JP)

(72) Inventor: Satoshi MATSUBARA, Tokyo (JP)

(21) Appl. No.: 18/412,139

(22) Filed: Jan. 12, 2024

(30) Foreign Application Priority Data
Jan. 31, 2023 (JP) 2023-13184

(51) Int. Cl. G01N 23/083; A61B 6/00; A61B 6/40; G01N 23/04; G01N 23/18

(52) U.S. Cl. CPC G01N 23/083; A61B 6/40; A61B 6/56; G01N 23/04; G01N 23/18; G01N 2223/1016; G01N 2223/652

(57) ABSTRACT

Proposed are an X-ray transmission inspection apparatus and an X-ray transmission inspection method, in which transmission images with large difference between irradiation angles are obtained so that the depth of the position of a foreign object can be obtained with high precision. The X-ray transmission inspection apparatus includes an X-ray source, an X-ray sensor configured to detect a transmitted X-ray, a moving mechanism configured to move the sample, and a calculation part configured to calculate a height position of a foreign object in a thickness direction in the sample, wherein the X-ray source performs irradiation with X-rays in a direction tilted with respect to the thickness direction of the sample and a transport direction of the sample, and the moving mechanism is capable of moving the sample in a first transport direction and in a second transport direction, and of changing X-ray irradiation angles.

### CROSS REFERENCE TO RELATED APPLICATION

The present application claims priority to Japan Patent Application No. 2023-13184, filed Jan. 31, 2023, the entire contents of which are incorporated herein for all purposes by this reference.

### BACKGROUND OF THE INVENTION

### Field of the Invention

The present disclosure relates to an X-ray transmission inspection apparatus and an X-ray transmission inspection method, in which a metal foreign object in a sample can be detected.

### Description of the Related Art

A lithium-ion secondary battery, which has a higher energy density than a nickel-metal hydride battery, is a type of non-aqueous electrolyte secondary battery in which lithium ions in an electrolyte are responsible for electrical conduction and metallic lithium is not included. The lithium-ion secondary battery is used as a battery for vehicles, hybrid vehicles and electric vehicles, as well as personal computers and mobile phones.

Generally, X-ray transmission inspection performed by an X-ray transmission image obtained by irradiating a sample with X-rays to detect a metal foreign object in the sample is used. For example, the X-ray transmission inspection is used to inspect a foreign object in a material of the lithium ion secondary battery.

For example, as described in a conventional technology disclosed in Patent Document 1, a foreign object detection method in which the presence or absence of a foreign object is detected by a transmitted X-ray image has been proposed.

In addition, as described in Patent Document 2, an image processing device is known in which a plurality of X-ray sensors is used to obtain a plurality of images from X-rays passing through a sample, and the amount of misalignment of the plurality of images is used so that a distance between an X-ray source and the sample, that is, depth information can be obtained.

### Documents of Related Art

(Patent Document 1) Japan Patent No. 5813923
(Patent Document 2) Japanese patent application publication No. Hei Sei 5-034131

### SUMMARY OF THE INVENTION

The related art described above has the problem described below.

In a conventional device described in Patent Document 2, although a plurality of X-ray sensors is used, a distance between the X-ray sensors is small, and change in an irradiation angle is small, and thus the amount of misalignment of a foreign object in a sample between transmission images decreases, so there is a problem that the estimation precision of the depth of the position of the foreign object is low.

The present disclosure is made in consideration of the above-mentioned problem, and is intended to provide an X-ray transmission inspection apparatus and an X-ray transmission inspection method, in which transmission images with large difference between irradiation angles are obtained so that the depth of the position of a foreign object can be obtained with high precision.

The present disclosure employs the following configurations to solve the above problems. That is, an X-ray transmission inspection apparatus according to a first invention includes: an X-ray source configured to irradiate a sample with an X-ray: an X-ray sensor installed on a side opposite to the X-ray source with respect to the sample and configured to detect transmitted X-rays when the X-rays pass through the sample: a moving mechanism configured to move the sample relative to the X-ray source and the X-ray sensor; and a calculation part configured to calculate a height position of a foreign object in a thickness direction in the sample based on the transmitted X-rays detected by the X-ray sensor, wherein the X-ray source performs irradiation with X-rays in a direction tilted with respect to the thickness direction of the sample and a transport direction of the sample, and the moving mechanism is capable of moving the sample in a first transport direction orthogonal to the thickness direction and in a second transport direction opposite to the first transport direction, and is capable of changing an X-ray irradiation angle for the sample when the moving mechanism moves the sample in the first transport direction and an X-ray irradiation angle for the sample when the moving mechanism moves the sample in the second transport direction by moving the X-ray source, the X-ray sensor, and the sample relative to one another.

In the X-ray transmission inspection apparatus, the moving mechanism may change an X-ray irradiation angle for the sample when the moving mechanism moves the sample in the first transport direction, and an X-ray irradiation angle for the sample when the moving mechanism moves the sample in the second transport direction by moving the X-ray source, the X-ray sensor, and the sample relative to one another, so transmission images with large difference between X-ray irradiation angles are obtained. Accordingly, between the different transmission images caused by irradiation with X-rays performed at different angles, an amount of misalignment of a foreign object in the sample increases at the depth position (the height position of the depth direction) of the foreign object, so the depth of the position of the foreign object may be estimated with high precision.

An X-ray transmission inspection apparatus according to a second invention features that in the first invention, the calculation part may calculate the height position of the foreign object by using an amount of misalignment between an X-ray transmission image detected when the sample is moved in the first transport direction and an X-ray transmission image detected when the sample is moved in the second transport direction.

An X-ray transmission inspection apparatus according to a third invention features that in the first or second invention, the X-ray sensor may be a TDI sensor.

An X-ray transmission inspection apparatus according to a fourth invention features that in the first or second invention, the moving mechanism may be capable of changing the X-ray irradiation angles by moving at least the X-ray source.

That is, in the X-ray transmission inspection apparatus, since the moving mechanism can change the irradiation angle of an X-ray by moving at least the X-ray source, the moving mechanism may move the X-ray source when moving the sample in the first transport direction and in the second transport direction so that the irradiation angle of the X-ray for the sample can be easily and greatly changed.

An X-ray transmission inspection apparatus according to a fifth invention features that the first or second invention, the moving mechanism may be capable of rotating the sample 180° with respect to a rotation axis according to an inspection width direction or a rotation axis according to the transport direction.

That is, in the X-ray transmission inspection apparatus, the moving mechanism may rotate the sample 180° with respect to the rotation axis according to the inspection width direction or the rotation axis according to the transport direction. Accordingly, when the sample is moved in the first transport direction and in the second transport direction, the sample may be rotated 180°, and thus without moving the X-ray source, the irradiation angle of an X-ray for the sample may be easily changed.

In addition, when the sample is rotated 180° with respect to the rotation axis according to the inspection width direction or the rotation axis according to the transport direction, the sample may be turned over, and a positive or negative sign of the irradiation angle of an X-ray for the sample may also be reversed.

An X-ray transmission inspection apparatus according to a sixth invention features that in the first invention, the apparatus further may include a reference piece installed on a surface of the moving mechanism or the sample, wherein the calculation part may calculate the height position of the foreign object by comparing the height position of the foreign object with a height position of the reference piece.

That is, in the X-ray transmission inspection apparatus, the calculation part may calculate the height position of the foreign object by comparing the height position of the foreign object with the height position of the reference piece. Accordingly, when the reference of a distance between a transmission image and a foreign object is not determined or when the bending of the sample is large, the height position of the foreign object may be compared with the reference piece on the surface of the moving mechanism or the sample detected, so the depth position (the height position) of the foreign object from the surface of the moving mechanism or the sample may be obtained.

An X-ray transmission inspection method according to a seventh invention using the X-ray transmission inspection apparatus of the first invention includes: a first movement step moving the sample in the first transport direction: a first passing point detection step detecting a position, as a first passing point, in the first transport direction at which the foreign object in the sample is detected based on transmitted X-rays detected by the X-ray sensor during the first movement step: a second movement step moving the sample in the second transport direction: a second passing point detection step detecting a position, as a second passing point, in the second transport direction, at which the foreign object in the sample is detected based on transmitted X-rays detected by the X-ray sensor during the second movement step; and a calculation step calculating a height position of the foreign object in the sample based on the first passing point, the second passing point, the X-ray irradiation angle when the sample is moved in the first transport direction, and the X-ray irradiation angle when the sample is moved in the second transport direction.

According to the present disclosure, the following effects are obtained.

That is, according to the X-ray transmission inspection apparatus and X-ray transmission inspection method according to the present disclosure, the moving mechanism can change an irradiation angle of an X-ray for a sample when the moving mechanism moves the sample in the first transport direction and an irradiation angle of an X-ray for the sample when the moving mechanism moves the sample in the second transport direction by moving the X-ray source, the X-ray sensor, and the sample relative to one another, and thus transmission images with large difference between irradiation angles of X-rays are obtained, and the amount of misalignment of an foreign object in the sample increases in a position of depth (a height position in a thickness direction) of the foreign object, thereby enabling the estimation of the depth of the position of a foreign object with high precision.

### BRIEF DESCRIPTION OF THE DRAWINGS

The above and other objectives, features, and other advantages of the present disclosure will be more clearly understood from the following detailed description when taken in conjunction with the accompanying drawings, in which:

FIGS. 1A, 1B, and 1C are schematic overall configuration views illustrating an inspection method in process order in a first embodiment of an X-ray transmission inspection apparatus and an X-ray transmission inspection method according to the present disclosure:

FIGS. 2A and 2B are views illustrating a relationship between the configuration of the apparatus and X-ray transmission images in an outward route (a first transport direction) of 2A and a return route (a second transport direction) of 2B in the first embodiment;

FIG. 3 is a schematic diagram illustrating a calculation method for obtaining the position of a foreign object in a height direction in the first embodiment:

FIG. 4 is a schematic diagram illustrating a reference piece and the height position of a foreign object when the sample is bent in the first embodiment;

FIG. 5 is a schematic diagram illustrating the height position of a foreign object when detecting the three-dimensional shape of the foreign object in the first embodiment:

FIGS. 6A and 6B are schematic diagrams illustrating an image example 6A of a first scan and an image example 6B of a second scan when detecting the three-dimensional shape of a foreign object in the first embodiment; and

FIGS. 7A, 7B and 7C are schematic overall configuration views illustrating an inspection method in process order in a second embodiment of the X-ray transmission inspection apparatus and X-ray transmission inspection method according to the present disclosure.

### DETAILED DESCRIPTION OF THE INVENTION

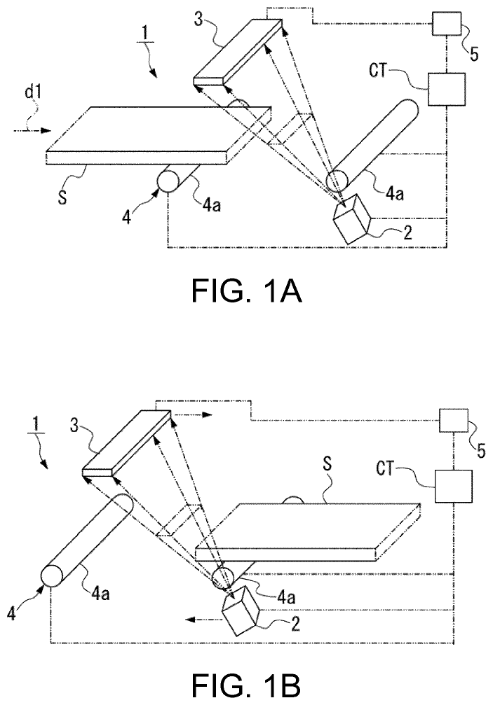

Hereinafter, a first embodiment of the X-ray transmission inspection apparatus and X-ray transmission inspection method according to the present disclosure will be described with reference to FIGS. 1A, 1B, 1C, 2A, 2B, 3, 4, 5, 6A, and 6B.

As illustrated in FIGS. 1A, 1B, and 1C, the X-ray transmission inspection apparatus 1 of this embodiment includes: an X-ray source 2 configured to irradiate a sample S with X-rays (a primary X-ray): an X-ray sensor 3 installed on a side opposite to the X-ray source 2 with respect to the sample S and configured to detect transmitted X-rays when the X-rays pass through the sample S; a moving mechanism 4 configured to move the sample S relative to the X-ray source 2 and the X-ray sensor 3; and a calculation part 5 configured to calculate a height position of a foreign object in a thickness direction in the sample S on the basis of the transmitted X-rays detected by the X-ray sensor 3.

The X-ray source 2 is disposed to perform irradiation with X-rays in a direction tilted with respect to the thickness direction of the sample S and the transport direction d1 or d2 (a direction orthogonal to the thickness direction of the sample S) of the sample S.

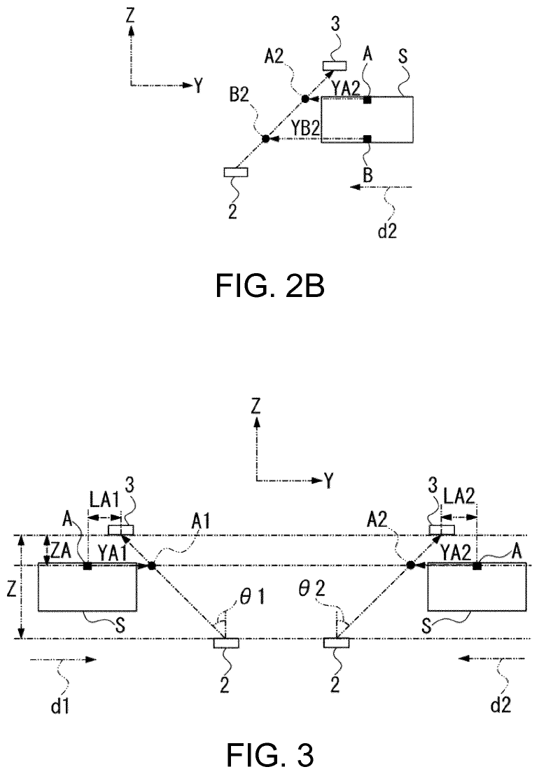

The moving mechanism 4 can move the sample S in a first transport direction d1 orthogonal to a thickness direction of the sample and can move the sample S in a second transport direction d2 opposite to the first transport direction d1, As illustrated in FIG. 3, the moving mechanism 4 can change an irradiation angle θ1 of an X-ray for the sample S when the moving mechanism 4 moves the sample S in the first transport direction d1, and an irradiation angle θ2 of an X-ray for the sample S when the moving mechanism 4 moves the sample S in the second transport direction d2 by moving the X-ray source 2, the X-ray sensor 3, and the sample S relative to one another.

The calculation part 5 has the function of calculating the height position of a foreign object by using the amount of misalignment between an X-ray transmission image detected when the sample S is moved in the first transport direction d1 and an X-ray transmission image detected when the sample S is moved in the second transport direction d2.

In this embodiment, the moving mechanism 4 can change an X-ray irradiation angle by moving at least the X-ray source 2.

That is, as illustrated in FIG. 1A, the X-ray source 2 and the X-ray sensor 3 are disposed by the moving mechanism 4 to perform irradiation with X-rays tilted with respect to the left when viewed from the front side toward the X-ray sensor 3 at the upper side from the X-ray source 2 at the lower side, and as illustrated in FIG. 1B, in this state, the sample S is moved in the first transport direction d1 and passes through the tilted X-ray.

Next, as illustrated in FIG. 1C, by using the moving mechanism 4, such as a motor, the X-ray source 2 is moved to the left side when viewed from the front side and the X-ray sensor 3 is moved to the right side when viewed from the front side, and thus the X-ray source 2 and the X-ray sensor 3 are disposed to perform irradiation with X-rays tilted with respect to the right side when viewed from the front side toward the X-ray sensor 3 at the upper side from the X-ray source 2 at the lower side. In this state, the sample S is moved in the second transport direction d2 opposite to the first transport direction d1 and passes through the X-ray tilted in a direction opposite to the direction in the case of the first transport direction d1. Accordingly, the moving mechanism 4 includes a device which reciprocates the sample S, and a device which moves the X-ray source 2 and the X-ray sensor 3 relative to one another.

In addition, as described above, in the case of the second transport direction d2, both the X-ray source 2 and the X-ray sensor 3 are moved, but only the X-ray source 2 may be moved and disposed to perform irradiation with X-rays in a direction tilted different from the irradiation direction in the case of the first transport direction d1.

In addition, an X-ray in the case of the first transport direction d1 (an outward route) and an X-ray in the case of the second transport direction d2 (a return route) are preset to intersect with each other.

In addition, although the X-ray in the case of the first transport direction d1 (the outward route) and the X-ray in the case of the second transport direction d2 (the return route) do not intersect with each other, reciprocating inspection positions may be misaligned in the transport direction as long as the position of an inspection width direction X is the same.

In addition, the X-ray transmission inspection apparatus 1 of this embodiment includes a control device CT that controls the X-ray source 2, the X-ray sensor 3, the moving mechanism 4, and the calculation part 5.

In addition, in this embodiment, the transport direction of the sample S is Y, the height direction (a depth direction) thereof is Z, and the inspection width direction thereof is X.

For example, the sample S is a strip-shaped material for lithium-ion batteries or a material used in the pharmaceutical industry.

The X-ray source 2, which is an X-ray tube capable of performing irradiation with X-rays, emits, from a window of beryllium foil, X-rays generated when hot electrons generated from a filament (cathode) in the tube are accelerated by a voltage applied between the filament (cathode) and a target (anode) and collide with tungsten (W), molybdenum (Mo), and chromium (Cr) of the target.

In addition, the X-ray transmission inspection apparatus 1 of this embodiment may include a collimator that limits the spread of X-rays from the X-ray source 2, or a polycapillary that converts X-rays into parallel X-rays.

The X-ray sensor 3 is a line sensor, such as a time delay integration (TDI) sensor, extending in a perpendicular direction (an inspection width direction X) with respect to the transport directions d1 and d2 in which a sample S moves.

A TDI sensor has a plurality of cells (sensor elements) disposed on a surface facing a corresponding X-ray source, and is provided with a fluorescent body disposed on a detection surface, a fiber optic plate (FOP) in which a plurality of optical fibers are arranged in multiple rows vertically and horizontally in a two-dimensional manner under the fluorescent body, and a Si light-receiving element disposed under the FOP. The TDI sensor has the same configuration as line sensors arranged in multiple rows.

For example, in the X-ray sensor 3, the TDI sensor is configured by 200 to 1000 unit line sensors lined up in the transport direction of the sample S.

The control device CT is a computer including a CPU which is connected to and controls the X-ray source 2, the X-ray sensor 3, the moving mechanism 4, and the calculation part 5.

This control device CT adjusts the direction and speed of charge transfer of the X-ray sensor 3, which is the TDI sensor, to the moving direction and speed of the sample S, and has the function of integrating the luminance values of X-rays received by the X-ray sensor 3 in the detection area of a light-receiving surface.

In addition, the control device CT may include the calculation part 5.

The moving mechanism 4 is, for example, a motor that can move relative to the X-ray sensor 3 in a direction in which a sheet-shaped sample S extends. For example, the moving mechanism 4 includes at least one pair of rollers 4a configured to move a strip-shaped sample S in an extension direction thereof in a roll-to-roll manner.

The calculation part 5 has a function of obtaining a contrast image from a transmission image representing the distribution of the intensity of detected transmitted X-rays.

As illustrated in FIGS. 1A, 1B, 1C, 2A, 2B, and 3, the X-ray transmission inspection method using the X-ray transmission inspection apparatus 1 of this embodiment includes: a first movement for moving a sample S in the first transport direction d1; detecting a position of the first transport direction d1 at which a foreign object in the sample S is detected, as a first passing point A1 on the basis of transmitted X-rays detected by the X-ray sensor 3 during the first movement: a second movement for moving the sample S in the second transport direction d2; detecting a position of the second transport direction d2, at which a foreign object in the sample S is detected, as a second passing point A2 on the basis of transmitted X-rays detected by the X-ray sensor 3 during the second movement; and calculating a height position of the foreign object in the sample S on the basis of the first passing point A1, the second passing point A2, the irradiation angle θ1 when the sample is moved in the first transport direction d1, and the irradiation angle θ2 when the sample is moved in the second transport direction.

Hereinafter, the X-ray transmission inspection method will be described by taking an example of a foreign object A present at a surface side (a side of the X-ray sensor 3) of the sample S, and a foreign object B present at another surface side (a side of the X-ray source 2) of the sample S.

First, in the first movement, when the sample S reaches an imaging start position (a detection start position) of the first transport direction d1, the output of a transmission image starts. In this output transmission image, the beginning of the first transport direction d1 is taken as the start of imaging (a reference point).

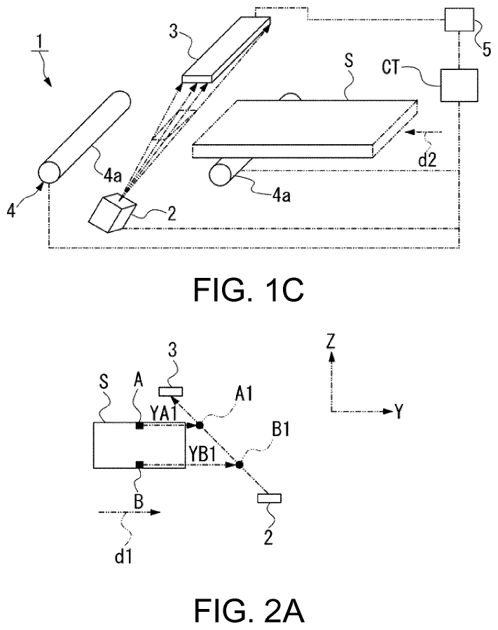

As illustrated in FIGS. 2A and 2B, in the detecting of the first passing point and the detecting of the second passing point, in the outward route in which the sample S moves in the first transport direction d1, a position at which the foreign object A passes through the ray axis of the X-ray source 2 and the X-ray sensor 3 is preset as the first passing point A1, and in the return route in which the sample S moves in the second transport direction d2, a position at which the foreign object A passes through the ray axis of the X-ray source 2 and the X-ray sensor 3 is preset as the second passing point A2.

In addition, in the outward route in which the sample S moves in the first transport direction d1, a position at which the foreign object B passes through the ray axis of the X-ray source 2 and the X-ray sensor 3 is preset as a first passing point B1, and in the return route in which the sample S moves in the second transport direction d2, a position at which the foreign object B passes through the ray axis of the X-ray source 2 and the X-ray sensor 3 is preset as a second passing point B2.

In addition, a distance between the foreign object A and the first passing point A1 is preset as YA1, a distance between the foreign object A and the second passing point A2 is preset as YA2, a distance between the foreign object B and the first passing pint B1 is preset as YB1, and a distance between the foreign object B and the second passing point B2 is preset as YB2.

As for a distance between a foreign object and the start of imaging, both in the first transport direction d1 (the outward route) and the second transport direction d2 (the return route), the foreign object B located on a surface of the side of the X-ray source 2 is farther from the start imaging than the foreign object A located on a surface of the side of the X-ray sensor 3 (YA1<YB1, YA2<YB2).

Accordingly, according to the height position of the thickness direction of the sample S containing foreign objects mixing with each other, timing at which a foreign object passes through the ray axis of an X-ray changes, and thus the height position of the thickness direction of the sample S containing the foreign objects mixing with each other can be estimated from difference of a distance between a reference point of the transmission image such as the start of imaging and a foreign object.

That is, calculation for estimating the height of each of foreign objects A and B mixed with each other from difference of a distance between a certain reference point and each of the foreign objects A and B on the basis of the transmission images of the X-ray sensor 3 in the calculating will be described by using FIG. 3. In addition, this calculation is performed by the calculation part 5.

The imaging start position is a position at which an image output starts on the basis of an arbitrarily set timing so that the inspection region of the sample S is output as a transmission image. A distance between the X-ray source 2 and a foreign object may be calculated by the following setting.

In addition, hereinafter, the case of moving in the first transport direction d1 is referred to as the outward route, and the case of moving in the second transport direction d2 is referred to as the return route.

In the outward route, an X-ray irradiation angle from the X-ray source 2 to the X-ray sensor 3:θ1

In the return route, an X-ray irradiation angle from the X-ray source 2 to the X-ray sensor 3: θ2

In the outward route, a distance in the height direction from the X-ray sensor 3 to the foreign object A: ZA1

In the return route, a distance in the height direction from the X-ray sensor 3 to the foreign object A: ZA2

Here, ZA1=ZA2=ZA.

In the outward route, a distance in the transport direction from the foreign object A to the start of imaging of the X-ray sensor 3: LA1

In the return route, a distance in the transport direction from the foreign object A to the start of imaging of the X-ray sensor 3: LA2

In the outward route, a distance in the transport direction from the foreign object A to the start of imaging of the X-ray sensor 3: YA1

In the return route, a distance in a transport direction from the foreign object A to the start of imaging of the X-ray sensor 3: YA2

$$YA 1 = ZA \cdot tan θ1 + LA 1$$

$$YA 2 = ZA \cdot tan θ2 + LA 2$$

Accordingly,

$$YA 1 + YA 2 = ( tan θ1 + tan θ2 ) ZA + ( LA 1 + LA 2 ) \qquad Equation 1$$

The value of YA1+YA2 increases in proportion to ZA.

Therefore, by obtaining the value of YA1+YA2 from X-ray transmission images, a distance from the X-ray source 2 to a foreign object A or B can be calculated.

In addition, in the case of |θ1|×|θ2|, as the irradiation angle increases, the estimation precision of the height position is improved.

For example, in a state in which the resolution is 10 μm, when |θ1|−|θ2|=45° (θ1=45°, θ2=45°), the estimation precision of the height position is +10 μm, and when |θ1|=|θ2|=30° (θ1=−30°, θ2=30°), the estimation precision of the height position is #17.3 μm.

In addition, in the technology of Patent Document 2, change in an irradiation angle is small, so the estimation precision of a height position is low.

The equation 1 is modified to the following equation, and the estimation accuracy of a height position decreases as an irradiation angle decreases.

$${Y}_{A 1} + {Y}_{A 2} = ( tan {θ}_{1} + tan {θ}_{2} ) {Z}_{A} + ( {L}_{A 1} + {L}_{A 2} ) ⇔ {Z}_{A} = \frac{{Y}_{A 1} + {Y}_{A 2}}{tan {θ}_{1} + tan {θ}_{2}} - \frac{{L}_{A 1} + {L}_{A 2}}{tan {θ}_{1} + tan {θ}_{2}} \qquad [ Equation 1 ]$$

When a precision of each of a distance YA1 and a distance YA2 is each #1 pixel, YA1+YA2 contains an error of up to ±2 pixel.

When resolution is set to 10 μm, the maximum error is ±20 μm.

When the error is included in the equation, the equation becomes the following equation.

$${Z}_{A} = \frac{{Y}_{A 1} + {Y}_{A 2}}{tan {θ}_{1} + tan {θ}_{2}} - \frac{{L}_{A 1} + {L}_{A 2}}{tan {θ}_{1} + tan {θ}_{2}} \pm \frac{20}{tan {θ}_{1} + tan {θ}_{2}} \qquad [ Equation 2 ]$$

For example, the error in the case of |θ1|−|θ2|−45° is calculated as follows.

$$\pm \frac{20}{tan {θ}_{1} + tan {θ}_{2}} \pm \frac{20}{1 + 1} \pm 10 \qquad [ Equation 3 ]$$

For example, the error in the case of |θ1|−|θ2|=30° is calculated as follows.

$$\pm \frac{20}{tan {θ}_{1} + tan {θ}_{2}} \pm \frac{20}{0.6 + 0.6} \pm 17.3 \qquad [ Equation 4 ]$$

In addition, since in Patent Document 2, an irradiation angle is stated as only a small angle, the assumed angle cannot be known. However, when |θ1|−|θ2|−1° is assumed and the error of the estimation accuracy of a height position is calculated under the same condition, the error is ±573 μm.

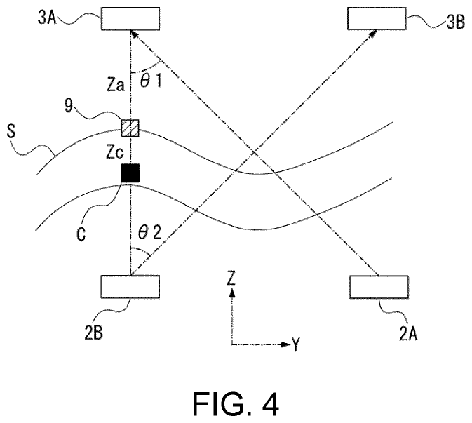

Next, when the reference of a distance between a transmission image and a foreign object is not determined or when the bending of the sample S is large, a reference piece 9 is placed on the surface of the sample S as illustrated in FIG. 4, and thus the depth position of the foreign object C from the surface of the sample S can be estimated.

That is, when due to the instability of the imaging start position, the imaging start position cannot be used as a reference of a distance up to a foreign object, when a feature point which is a reference from a transmission image, such as the end of the sample S, cannot be determined, or when a distance between the X-ray sensor 3 and the surface of the sample S is not constant due to the bending of the sample S, the depth position of the foreign object can be estimated.

In the X-ray transmission inspection apparatus 1 of this embodiment, when the sample S is bent, the reference piece 9 installed on the surface of the sample S is provided as illustrated in FIG. 4, and thus the calculation part 5 can calculate the height position of the foreign object C by comparing the height position of the foreign object C with the height position of the reference piece 9.

With an X-ray source 2A and an X-ray sensor 3A disposed in the first transport direction d1 (the outward route) and with an X-ray source 2B and an X-ray sensor 3B disposed in the second transport direction d2 (the return route), for example, when a distance between the X-ray sensor 3A and the reference piece 9 (the surface of the sample S) of metal is Za, and a distance between the X-ray sensor 3A and the foreign object C is Zc, a distance between the reference piece 9 (the surface of the sample S) and the foreign object C is Za-Zc.

In addition, the reference piece 9 may be installed on a surface of a component of the moving mechanism 4 close to the sample S.

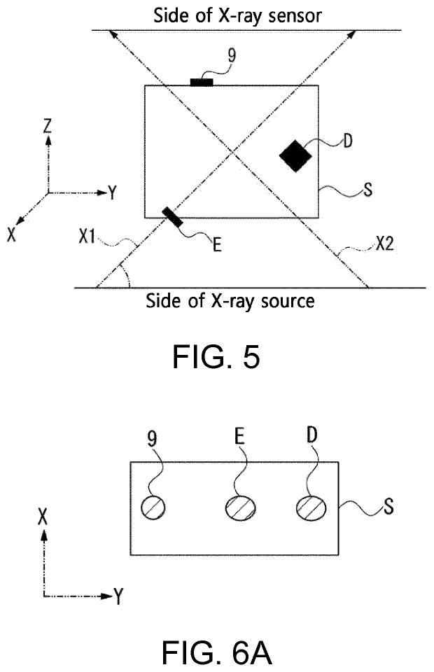

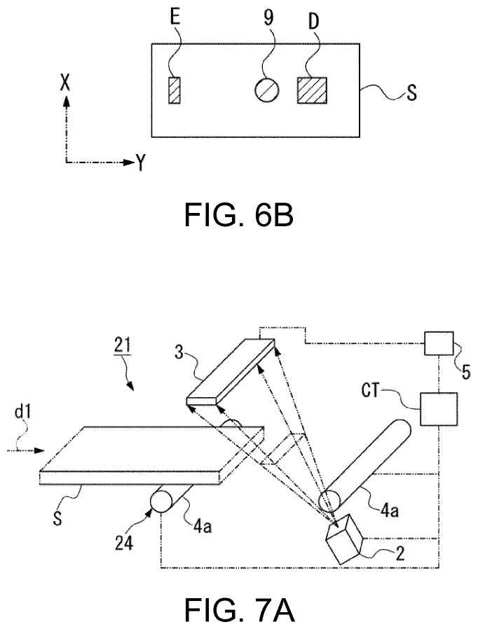

Next, in the X-ray transmission inspection apparatus 1 of this embodiment, as illustrated in FIGS. 5, 6A, and 6B, by integrating information on the sizes and shapes of foreign objects calculated from a plurality of two-dimensional images, the estimation precision of the three-dimensional shapes of the foreign objects D and E is improved.

That is, for example, as illustrated in FIG. 5, in a case in which foreign objects D and E are present in the sample S and the reference piece 9 is installed on the surface (a side of the X-ray sensor) of the sample S, a transmission image detected by an X-ray X1 of the outward route (at a first scan) is illustrated in FIG. 6A, and a transmission image detected by an X-ray X2 of the return route (at a second scan) is illustrated in FIG. 6B.

Accordingly, it is possible to obtain information on the sizes and shapes of the foreign objects D and E from a plurality of two-dimensional images (the transmission image of the outward route and the transmission image of the return route).

Accordingly, in the X-ray transmission inspection apparatus 1 of this embodiment, the moving mechanism 4 can change an X-ray irradiation angle for the sample S when the moving mechanism 4 moves the sample S in the first transport direction d1, and an X-ray irradiation angle for the sample S when the moving mechanism 4 moves the sample S in the second transport direction d2 by moving the X-ray source 2, the X-ray sensor 3, and the sample S relative to one another, so transmission images with large difference between X-ray irradiation angles θ1 and θ2 are obtained. Accordingly, between the different transmission images caused by irradiation with X-rays performed at different angles θ1 and θ2, the amount of misalignment of the foreign object in the sample S increases at the depth position (the height position of the depth direction) of the foreign object, so the depth of the position of the foreign object can be estimated with high precision.

In addition, since the moving mechanism 4 can change the irradiation angles of an X-ray by moving at least the X-ray source 2, the moving mechanism 4 can move the X-ray source 2 when moving the sample S in the first transport direction d1 and in the second transport direction d2 so that the irradiation angles of the X-ray for the sample S can be easily and greatly changed.

In addition, the calculation part 5 calculates the height position of a foreign object by comparing the height position of a foreign object with the height position of the reference piece 9. Accordingly, when the reference of a distance between a transmission image and a foreign object is not determined or when the bending of the sample S is large, the height position of a foreign object may be compared with the reference piece 9 on the surface of the sample S detected, so the depth position (the height position) of the foreign object from the surface of the sample foreign object may be obtained.

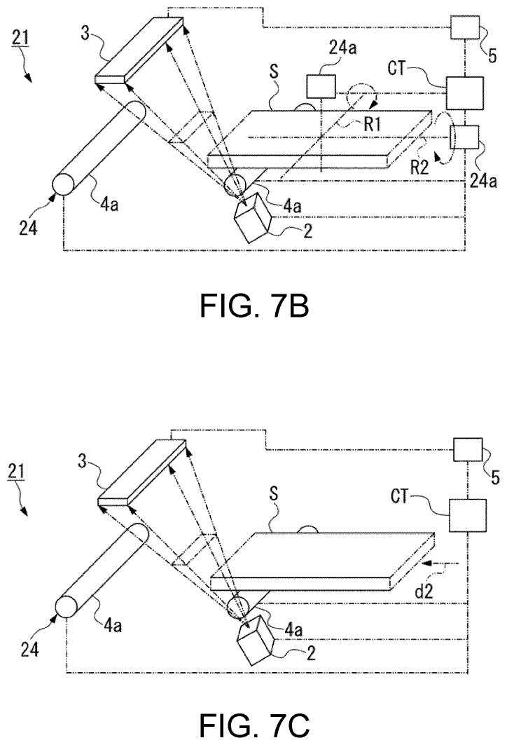

Next, a second embodiment of the X-ray transmission inspection apparatus and X-ray transmission inspection method according to the present disclosure will be described with reference to FIGS. 7A, 7B, and 7C below. In addition, in the description of the following embodiment, the same components described in the above embodiment are given the same reference numerals, and descriptions thereof are omitted.

Difference between the second embodiment and the first embodiment will be described. In the first embodiment, the moving mechanism 4 changes the irradiation angle of X-ray by moving the X-ray source 2 and the X-ray sensor 3, but in the X-ray transmission inspection apparatus 21 of the second embodiment, as illustrated in FIGS. 7A, 7B, and 7C, a moving mechanism 24 includes a rotation device 24a which can rotate the sample S 180° with respect to a rotation axis R1 according to the inspection width direction or a rotation axis R2 according to the transport direction d1 or d2.

In the second embodiment, as illustrated in 7A, irradiation with X-rays is generated from the X-ray source 2 toward the X-ray sensor 3, and the sample S is moved in the first transport direction d1 (the outward route) by the moving mechanism 24 and passes through the tilted X-ray to detect a transmission image.

Next, as illustrated in 7B, the sample S is rotated 180° with respect to the rotation axis R1 according to the inspection width direction or the rotation axis R2 according to the transport direction d1 or d2 by a motor of the rotation device 24a of the moving mechanism 24.

After the sample S is rotated, as illustrated in 7C, the sample S is moved in the second transport direction d2 (the return route) and passes through the tilted X-ray to detect a transmission image. In addition, in the second embodiment, the sample S is rotated, but the arrangement of the X-ray source 2 and the X-ray sensor 3 is not changed.

Based on transmission images obtained before and after the rotation of the sample S (in the first transport direction d1 and the second transport direction d2), the height position of a foreign object can be calculated by the calculation part 5 in the same manner as the first embodiment.

That is, when the sample S is rotated 180° with respect to the rotation axis R1 according to the inspection width direction or the rotation axis R2 according to the transport direction, the sample S is turned over and the positive or negative sign of the irradiation angle of an X-ray for the sample S is also reversed.

Accordingly, in the X-ray transmission inspection apparatus 21 of the second embodiment, the moving mechanism 24 can rotate the sample S 180° with respect to the rotation axis R1 according to the inspection width direction or the rotation axis R2 according to the transport direction d1 or d2, and thus when the sample S is moved in the first transport direction d1 and in the second transport direction d2, the sample S is rotated 180°, so the irradiation angle of an X-ray for the sample S can be easily changed without moving the X-ray source 2.

In addition, the technical scope of the present disclosure is not limited to the above-described embodiments, and various changes may be made without departing from the spirit of the present disclosure.

What is claimed is:

1. An X-ray transmission inspection apparatus comprising:
an X-ray source configured to irradiate a sample with X-rays:
an X-ray sensor installed on a side opposite to the X-ray source with respect to the sample and configured to detect transmitted X-rays when X-rays pass through the sample:
a moving mechanism configured to move the sample relative to the X-ray source and the X-ray sensor; and
a calculation part configured to calculate a height position of a foreign object in a thickness direction in the sample based on the transmitted X-rays detected by the X-ray sensor,
wherein the X-ray source performs irradiation with X-rays in a direction tiled with respect to the thickness direction of the sample and a transport direction of the sample, and
the moving mechanism is capable of moving the sample in a first transport direction orthogonal to the thickness direction and in a second transport direction opposite to the first transport direction, and is capable of changing an X-ray irradiation angle for the sample when the moving mechanism moves the sample in the first transport direction and an X-ray irradiation angle for the sample when the moving mechanism moves the sample in the second transport direction by moving the X-ray source, the X-ray sensor, and the sample relative to one another.

2. The X-ray transmission inspection apparatus according to claim 1, wherein the calculation part calculates the height position of the foreign object by using an amount of misalignment between an X-ray transmission image detected when the sample is moved in the first transport direction and an X-ray transmission image detected when the sample is moved in the second transport direction.

3. The X-ray transmission inspection apparatus according to claim 1, wherein the X-ray sensor is a TDI sensor.

4. The X-ray transmission inspection apparatus according to claim 2, wherein the X-ray sensor is a TDI sensor.

5. The X-ray transmission inspection apparatus according to claim 1, wherein the moving mechanism is capable of changing the X-ray irradiation angles by moving at least the X-ray source.

6. The X-ray transmission inspection apparatus according to claim 2, wherein the moving mechanism is capable of changing the X-ray irradiation angles by moving at least the X-ray source.

7. The X-ray transmission inspection apparatus according to claim 1, wherein the moving mechanism is capable of rotating the sample 180° with respect to a rotation axis according to an inspection width direction or a rotation axis according to the transport direction.

8. The X-ray transmission inspection apparatus according to claim 2, wherein the moving mechanism is capable of rotating the sample 180° with respect to a rotation axis according to an inspection width direction or a rotation axis according to the transport direction.

9. The X-ray transmission inspection apparatus according to claim 1, further comprising:
a reference piece installed on a surface of the moving mechanism or the sample,
wherein the calculation part calculates the height position of the foreign object by comparing the height position of the foreign object with a height position of the reference piece.

10. An X-ray transmission inspection method using the X-ray transmission inspection apparatus according to claim 1, the method comprising:
a first movement step moving the sample in the first transport direction;
a first passing point detection step detecting a position, as a first passing point, in the first transport direction at which a foreign object in the sample is detected based on transmitted X-rays detected by the X-ray sensor during the first movement step;
a second movement step moving the sample in the second transport direction;
a second passing point detection step detecting a position, as a second passing point, in the second transport direction at which the foreign object in the sample is detected based on transmitted X-rays detected by the X-ray sensor during the second movement step; and
a calculation step calculating a height position of the foreign object in the sample based on the first passing point, the second passing point, the X-ray irradiation angle when the sample is moved in the first transport direction, and the X-ray irradiation angle when the sample is moved in the second transport direction.
